(19) United States
(12) Patent Application Publication
(10) Pub. No.: US 2021/0093721 A1
(43) Pub. Date: Apr. 1, 2021

(54) COLLOIDAL PARTICLES FOR USE IN MEDICINE

(71) Applicant: Cantab Biopharmaceuticals Patents Limited, Valetta (MT)

(72) Inventors: William HENRY, London (GB); Richard WOLF-GARRAWAY, London (GB); John Charles MAYO, London (GB)

(21) Appl. No.: 16/883,338

(22) Filed: May 26, 2020

Related U.S. Application Data
(63) Continuation of application No. 15/768,381, filed on Apr. 13, 2018, now abandoned.

(30) Foreign Application Priority Data
Oct. 14, 2015 (GB) 1518172.0

(51) Int. Cl. A61K 47/24; A61K 9/00; A61K 9/127; A61P 7/00; A61P 5/00; A61K 47/10

(52) U.S. Cl. CPC A61K 47/24; A61K 9/0019; A61K 9/1271; A61K 47/10; A61P 7/00; A61P 5/00; A61K 9/0014

(57) ABSTRACT

The invention provides a composition comprising a colloidal particle comprising about 0.5 to 20 mole percent of an amphipathic lipid derivatized with a biocompatible hydrophilic polymer for use in medicine, wherein said composition does not contain any pharmaceutically active agent.

### CROSS-REFERENCES TO RELATED APPLICATIONS

This application is a continuation of U.S. application Ser. No. 15/768,381, filed Apr. 13, 2018, which is a National Stage Entry of PCT International Application No. PCT/EP2016/74759, filed on Oct. 14, 2016, which claims priority to GB Application No. 1518172.0, filed Oct. 14, 2015, the contents of which are incorporated herein by reference in their entirety.

The present invention relates to colloidal particles for use in medicine.

Many diseases in patients are characterised by sub-optimal levels in the blood or extracellular environment of an endogenous factor or hormone. The patient has a low level of the circulating endogenous factor or hormone which is insufficient to provide the necessary level of biological action or signalling to cells, tissues or organs. The patient may be characterised as suffering from a mild form of the disease in question.

For example, individuals with less than 1% active Factor VIII can be classified as having severe haemophilia A, those with 1-5% active Factor VIII as having moderate haemophilia A, and those with between 5-40% of normal levels of active clotting Factor VIII as having mild haemophilia A.

Diseases which are characterised by the absence of or an insufficient amount of an endogenous factor or hormone are traditionally treated by a replacement therapy in which the patient receives regular doses of the factor or hormone. However, subjects with mild to moderate forms of the disease do not require the same dosages of the therapeutic composition used to treat such disorders as for patients with none or almost none of the endogenous factor or hormone in question.

As in the treatment of any disease it is important to avoid prescribing a patient with too high a dose as many therapeutic agents can give rise to significant toxicities. Other problems with replacement therapy include the development of “resistance” through the formation of autoantibodies in the patient to the replacement factor or hormone.

Replacement therapies aim to restore biological function of the endogenous factor or hormone by administering an exogenous version of the factor or hormone which may be prepared by a variety of means, including chemical synthesis, recombinant DNA technology, donation from allogeneic donors or isolation from cadaveric sources. For example, the treatment of haemophilia relies on the administration of blood factors and the treatment of diabetes uses insulin. Such therapies have used a variety of sources for the agent in question with various different formulations and routes of administration. However, factors and hormones obtained from donor sources carries with it the risk of contamination and synthetic routes of preparation are costly.

An example of this approach can be seen in the treatment of haemophilia with exogenous blood factors. Typically, blood factors have been prepared as pharmaceutical compositions for intravenous administration. The compositions have been based on the active protein, often conjugated to a polymer such as polyethylene glycol (PEG) to improve the half-life in circulation. Intravenous administration of PEGylated blood factors as therapeutic agents is therefore well understood and widely accepted. Liposomal formulations of naked (i.e. unconjugated and without modification) blood factors such as Factor VIII and Factor IX substances are known also, see for example WO 95/04524.

Pharmaceutical compositions comprising Factor VIII and liposomes modified by the presence of polyethylene glycol are described in WO 99/55306 in which the blood factor is not encapsulated in the liposome. However, the formulations are prepared for intravenous administration. Additional formulations of other proteins are described in WO 2004/091723 where the proteins include blood clotting factors. The proteins are said to bind to the liposomes in a non-covalent manner through interaction with the polyethylene glycol present on the surface of the liposomes. However, the formulations of blood clotting factors prepared according to the examples of this document are also for intravenous administration.

Other examples of formulations of blood factors, Factor VIII and Factor VIIa, present as a conjugate with PEG are shown in WO 2011/135307 and WO 2011/135308 respectively where the actual formulations prepared were for intravenous administration. WO 2013/156488 also describes a dosage form of modified therapeutic agents, including blood factors such as Factor VIII (FVIII) and Factor VIIa (FVIIa), for subcutaneous administration.

The blood factor Factor VIII has also been found to be capable of association with PEGylated liposomes, i.e. the blood factor is not encapsulated inside the liposome (Baru et al Thromb. Haemost, 93, pages 1061-1068, (2005)). However, the compositions of FVIII were only prepared as formulations for intravenous administration.

Further studies by Peng et al in The AAPS Journal, 14(1), pages 25-42 (2011) disclose an alternative approach based on FVIII encapsulated in liposomes which are subsequently PEGylated by passively adding PEG to the liposomes after preparation. In one experiment in Peng et al the liposomal formulation is administered subcutaneously (SC) to investigate immunogenicity but there is no suggestion of a therapeutic purpose to this administration. In Peng et al there is also a specific reference to the paper of Baru et al (2005) and a statement that the approach of Baru et al “exposed FVIII to plasma components such as proteases and IgGs”. Liposomes prepared according to the method of Baru et al (2005) containing recombinant Factor VIII have been administered intravenously to subjects (Spira et al Blood, 108 (12), pages 3668-3673 (2006)).

It would, therefore, be preferable for a patient suffering from a mild to moderate form of such a disease to have a means of supplementing the endogenous levels of the factor or hormone in question in such a way that external sources of the factor or hormone were not necessarily required.

According to the present invention there is provided a composition comprising a colloidal particle comprising approximately 0.5 to 20 mole percent of an amphipathic lipid derivatized with a biocompatible hydrophilic polymer for use in medicine. The composition consists of the colloidal particles without any other pharmaceutically active agent being present. Such pharmaceutically active agents are defined as drugs, substances, molecules or compounds having a pharmacological effect on the body of a subject.

The composition comprising the colloidal particles of the invention may be used in the treatment of a disease in a subject characterised by insufficient levels of an endogenous factor or hormone in the subject. The disease may result from a loss of function or partial loss of function of a gene or genes connected with the production of the endogenous factor or hormone.

The compositions of the present invention therefore provide for the protection and prolongation of the activity of an endogenous biologically active polypeptide or protein. The invention provides for the therapy of a disease or a condition without the need for administration of exogenous protein with or without further derivatization. Where the composition is administered topically the invention also means that a subject is not required to receive any injections as in conventional therapies in order to maintain a therapeutically effective circulating level of the biologically active polypeptide or protein. If the subject is already regularly receiving an exogenous injection of a biologically active polypeptide or protein, the composition will prolong the life of such polypeptide or protein within the body enabling either the administered dose of such polypeptide or protein to be reduced, the intervals between doses to be increased or a combination of both these benefits.

The disease may be selected from the group consisting of a blood factor disease, an endocrine disorder or a hormone deficiency.

The blood factor disease may be a haemophilia (haemophilia A, B or C), von Willebrand Disease, Factor V deficiency, Factor X deficiency, Factor XII deficiency.

The endocrine disorder may be acromegaly, Addison's Disease, Cushing's Syndrome, De Quervain's Thyroiditis, obesity, diabetes mellitus (Type 1 diabetes or Type 2 diabetes), diabetes insipidous, Goiter, Graves' Disease, Growth Disorders, Growth Hormone Deficiency, Hashimoto's Thyroiditis, Hyperglycaemia, Hyperparathyroidism, Hypoglycaemia, Hypoparathyroidism, Low Testosterone, Menopause, Osteoporosis, Parathyroid Diseases, Pituitary Disorders, Polycystic Ovary Syndrome, Prediabetes, Turner Syndrome.

The colloidal particles may be substantially neutral and the polymer may carry substantially no net charge. The colloidal particles may have a mean particle diameter of between about 0.03 to about 0.4 microns (μm), for example having a mean particle diameter of approximately 0.1 microns (μm). A mean particle diameter in this range may increase the circulation time of the particles in vivo and prevent their adsorption by the reticuloendothelial system (RES).

The colloidal particles of the invention are typically in the form of lipid vesicles or liposomes as are well known in the art. References to colloidal particles in the present specification include liposomes and lipid vesicles unless the context specifies otherwise.

In the colloidal particles, the amphipathic lipid may be a phospholipid from natural or synthetic sources. The amphipathic lipid may comprise approximately 0.5 to about 20 mole percent (%) of the particles, for example approximately about 1 to 20%, or about 1 to 6%, or about 3%.

An “amphipathic lipid” refers to a substance including a hydrophilic region and a hydrophobic region, such as phospholipids. Amphipathic lipids can be zwitterionic phospholipids, zwitterionic lipids, lipids having a net negative charge, and lipids having a net positive charge. For example, amphipathetic lipids include, but are not limited to, phosphatidylcholines, phosphatidylserines, phosphatidylethanolamines, phosphatidylinositols, sphingomyelin, soybean lecithin (soya lecithin), egg lecithin, lysophosphatidylcholines, lysophosphatidylethanolamines, phosphatidylglycerols, phosphatidylserines, phosphatidylinositols, phosphatidic acids, cardiolipins, acyl trimethylammonium propane, diacyldimethylammonium propane, stearylamine, ethyl phosphatidylcholine and the like. Soya lecithin is a combination predominantly of naturally-occurring phospholipids; phosphatidylcholine (PC), phosphatidylethanolamine (PE) and phosphatidylinositol (PI).

Suitable examples of such amphipathic lipids include a phosphatidylethanolamine (PE), a carbamate-linked uncharged lipopolymer or aminopropanediol distearoyl (DS), or mixtures thereof.

Examples of phosphatidylethanolamine phospholipids include 1,2-Dierucoyl-sn-glycero-3-phosphoethanolamine, 1,2-Dilauroyl-sn-glycero-3-phosphoethanolamine, 1,2-Dimyristoyl-sn-glycero-3-phosphoethanolamine, 1,2-Dioleoyl-sn-glycero-3-phosphoethanolamine, 1,2-Dipalmitoyl-sn-glycero-3-phosphoethanolamine, 1,2-Distearoyl-sn-glycero-3-phosphoethanolamine, 1-Palmitoyl-2-oleoyl-sn-glycero-3-phosphoethanolamine. A suitable example of phosphatidyl ethanolamine (PE) may be 1,2-distearoyl-sn-glycero-3-phosphoethanol-amine (DSPE). The purpose of the biocompatible hydrophilic polymer is to sterically stabilize the SUVs, thus preventing fusion of the vesicles in vitro, and allowing the vesicles to escape adsorption by the RES in vivo.

The colloidal particles may further comprise a second amphipathic lipid obtained from either natural or synthetic sources. The second amphipathic lipid may be a phosphatidylcholine (PC). Examples of phosphatidylcholine phospholipids include 1,2-Didecanoyl-sn-glycero-3-phosphocholine, 1,2-Dierucoyl-sn-glycero-3-phosphocholine, 1,2-Dilinoleoyl-sn-glycero-3-phosphocholine, 1,2-Dilauroyl-sn-glycero-3-phosphocholine, 1,2-Dimyristoyl-sn-glycero-3-phosphocholine, 1,2-Dioleoyl-sn-glycero-3-phosphocholine, 1,2-Dipalmitoyl-sn-glycero-3-phosphocholine, 1,2-Distearoyl-sn-glycero-3-phosphocholine, 1-Myristoyl-2-palmitoyl-sn-glycero 3-phosphocholine, 1-Myristoyl-2-stearoyl-sn-glycero-3-phosphocholine, 1-Palmitoyl-2-myristoyl-sn-glycero-3-phosphocholine, 1-Palmitoyl-2-oleoyl-sn-glycero-3-phosphocholine, 1-Palmitoyl-2-stearoyl-sn-glycero-3-phosphocholine, 1-Stearoyl-2-myristoyl-sn-glycero-3-phosphocholine, 1-Stearoyl-2-oleoyl-sn-glycero-3-phosphocholine, 1-Stearoyl-2-palmitoyl-sn-glycero-3-phosphocholine. A suitable example of phosphatidyl choline (PC) may be palmitoyl-oleoyl phosphatidyl choline (POPC) or soy phosphatidyl choline. Other natural sources of phosphatidylcholine include egg phosphatidylcholine. The phosphatidylcholine may be hydrogenated or non-hydrogenated.

In one embodiment, the pharmaceutical composition may be composed of colloidal particles which comprise palmitoyl-oleoyl phosphatidyl choline (POPC) and 1,2-distearoyl-sn-glycero-3-phosphoethanol-amine (DSPE) in a molar ratio (POPC:DSPE) of from 85 to 99:15 to 1. In some cases, the molar ratio of POPC:DSPE may be from 90 to 99:10 to 1. In one embodiment, the molar ratio of POPC:DSPE may be 97:3. A molar ratio of 97:3 of these lipids is equivalent to a 97:10 weight ratio.

In an alternative embodiment, the pharmaceutical composition of the invention may be supplemented with cholesterol.

The biocompatible polymer may have a molecular weight of between about 500 to about 5000 Daltons, for example approximately 2000 Daltons.

The biocompatible hydrophilic polymer used according to the invention may be selected from the group consisting of polyalkylethers, polylactic acids and polyglycolic acids. The biocompatible hydrophilic polymer may be polyethylene glycol (PEG). The polyethylene glycol as used in the compositions of the invention may have a molecular weight of between about 500 to about 5000 Daltons, for example it may have a molecular weight of approximately 1000, 2000, or 3000 Daltons. In one embodiment the molecule weight of the PEG may be 2000 Daltons. The polyethylene glycol may be branched or unbranched.

An example of a suitable derivatized amphipathic lipid may be 1,2-distearoyl-sn-glycero-3-phosphoethanol-amine-N-[poly-(ethyleneglycol)]. If the PEG has a molecular weight of 2000 Daltons, the derivatized amphipathic lipid may be described as 1,2-distearoyl-sn-gly cero-3-phosphoethanol-amine-N-[poly-(ethylenegly col)-2000] (DSPE-PEG 2000).

The concentration of the derivatized amphipathic lipid in the final formulation of colloidal particles may be adjusted accordingly to suit the desired properties of the composition. However, suitable concentrations may be from 5.0 mg/mL to 15 mg/mL, for example 7.5 mg/mL to 12.5 mg/mL, or 7.6 mg/mL to 9.0 mg/mL. The overall content of lipid in the final formulation may be in the range of from 4% to 12%, for example 4%, 5%, 6%, 7%, 8%, 9%, 10%, 11% or 12%.

The composition may comprise any suitable excipient, buffer and/or adjuvant and may be formulated as a pharmaceutical composition. Examples of such excipients, buffers and/or adjuvants, include phosphate buffered saline (PBS), potassium phosphate, sodium phosphate and/or sodium citrate. Other biological buffers can include PIPES, MOPS etc.

Suitable pH values for the composition include any generally acceptable pH values for administration in vivo, such as for example pH 5.0 to pH 9.0, suitably from pH 6.8 to pH 7.2, or pH 7.0.

The present inventors have surprisingly found that formulations of colloidal particles (liposomes) derivatized with a biocompatible polymer can be successfully administered and achieve a therapeutically effective dose in a subject. Suitably, the biocompatible polymer is polyethylene glycol.

This aspect of the invention therefore extends to a method of treatment of a subject suffering from a disease or condition as defined above, comprising administering to the subject a composition comprising colloidal particles as defined above. The invention includes uses of such colloidal particles in the manufacture of a medicament for the treatment of such a disease or condition.

Without wishing to be bound by theory, it is believed that that the colloidal particles used in accordance with the present invention can potentiate the activity of a factor or hormone in a subject. In many diseases, the symptoms of the disease can be manifested when a subject has sub-optimal levels of the factor or hormone.

Where the factor is a blood factor it may be selected from the group consisting of Factor VII, Factor VIIa, Factor VIII, Factor IX, Factor X, Factor Xa, Factor XI, Factor VIIa, Factor V, Factor XIII, von Willebrand's Factor and Protein C. In some embodiments the blood coagulation factor may be suitably Factor VII, Factor VIII or Factor IX.

Other factors or hormones include but are not limited to, calcitonin, erythropoietin (EPO), granulocyte colony stimulating factor (GCSF), thrombopoietin (TPO), alpha-1 proteinase inhibitor, granulocyte macrophage colony stimulating factor (GMCSF), growth hormone, heparin, human growth hormone (HGH), growth hormone releasing hormone (GHRH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), insulin, pro-insulin, amylin, C-peptide, somatostatin, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide (GLP).

The factor may also be an antibody or an antibody fragment for example; a single-domain antibody, VL, VH, Fab, F(ab′)2, Fab′, Fab3, scFv, di-scFv, sdAb, Fc and/or combinations thereof.

A variety of known coupling reactions may be used for preparing vesicle forming lipids derivatized with hydrophilic polymers. For example, a polymer (such as PEG) may be derivatized to a lipid such as phosphatidylethanolamine (PE) through a cyanuric chloride group. Alternatively, a capped PEG may be activated with a carbonyl diimidazole coupling reagent, to form an activated imidazole compound. A carbamate-linked compound may be prepared by reacting the terminal hydroxyl of MPEG (methoxyPEG) with p-nitrophenyl chloroformate to yield a p-nitrophenyl carbonate. This product is then reacted with 1-amino-2,3-propanediol to yield the intermediate carbamate. The hydroxyl groups of the diol are acylated to yield the final product. A similar synthesis, using glycerol in place of 1-amino-2, 3-propanediol, can be used to produce a carbonate-linked product, as described in WO 01/05873. Other reactions are well known and are described, e.g. in U.S. Pat. No. 5,013,556.

Colloidal particles (liposomes) can be classified according to various parameters. For example, when the size and number of lamellae (structural parameters) are used as the parameters then three major types of liposomes can be described: Multilamellar vesicles (MLV), small unilamellar vesicles (SUV) and large unilamellar vesicles (LW).

MLV are the species which form spontaneously on hydration of dried phospholipids above their gel to liquid crystalline phase transition temperature (cm). The size of the MLVs is heterogeneous and their structure resembles an onion skin of alternating, concentric aqueous and lipid layers.

SUV are formed from MLV by sonication or other methods such as extrusion, high pressure homogenisation or high shear mixing and are single layered. They are the smallest species with a high surface-to-volume ratio and hence have the lowest capture volume of aqueous space to weight of lipid.

The third type of liposome LUV has a large aqueous compartment and a single (unilamellar) or only a few (oligolamellar) lipid layers. Further details are disclosed in D. Lichtenberg and Y. Barenholz, in “Liposomes: Preparation, Characterization, and Preservation, in Methods of Biochemical Analysis”, Vol. 33, pp. 337-462 (1988).

As used herein the term “loading” means any kind of interaction of the biopolymeric substances to be loaded, for example, an interaction such as encapsulation, adhesion (to the inner or outer wall of the vesicle) or embedding in the wall with or without extrusion of the biopolymeric substances.

As used herein and indicated above, the term “liposome” refers to colloidal particles and is intended to include all spheres or vesicles of any amphipathic compounds which may spontaneously or non-spontaneously vesiculate, for example phospholipids where at least one acyl group replaced by a complex phosphoric acid ester. The liposomes may be present in any physical state from the glassy state to liquid crystal. Most triacylglycerides are suitable and the most common phospholipids suitable for use in the present invention are the lecithins (also referred to as phosphatidylcholines (PC)), which are mixtures of the diglycerides of stearic, palmitic, and oleic acids linked to the choline ester of phosphoric acid. The lecithins are found in all animals and plants such as eggs, soybeans, and animal tissues (brain, heart, and the like) and can also be produced synthetically. The source of the phospholipid or its method of synthesis are not critical, any naturally occurring or synthetic phosphatide can be used.

Examples of specific phosphatides are L-a-(distearoyl) lecithin, L-a-(dipalmitoyl) lecithin, L-a-phosphatide acid, L-a-(dilauroyl)-phosphatidic acid, L-a(dimyristoyl) phosphatidic acid, L-a(dioleoyl)phosphatidic acid, DL-a (di-palmitoyl) phosphatidic acid, L-a(distearoyl) phosphatidic acid, and the various types of L-a-phosphatidylcholines prepared from brain, liver, egg yolk, heart, soybean and the like, or synthetically, and salts thereof. Other suitable modifications include the controlled peroxidation of the fatty acyl residue cross-linkers in the phosphatidylcholines (PC) and the zwitterionic amphipathates which form micelles by themselves or when mixed with the PCs such as alkyl analogues of PC.

The phospholipids can vary in purity and can also be hydrogenated either fully or partially. Hydrogenation reduces the level of unwanted peroxidation, and modifies and controls the gel to liquid/crystalline phase transition temperature (try) which effects packing and leakage.

The liposomes can be “tailored” to the requirements of any specific reservoir including various biological fluids, maintains their stability without aggregation or chromatographic separation, and remains well dispersed and suspended in the injected fluid. The fluidity in situ changes due to the composition, temperature, salinity, bivalent ions and presence of proteins. The liposome can be used with or without any other solvent or surfactant.

Generally suitable lipids may have an acyl chain composition which is characteristic, at least with respect to transition temperature (Tm) of the acyl chain components in egg or soybean PC, i.e., one chain saturated and one unsaturated or both being unsaturated. However, the possibility of using two saturated chains is not excluded.

The liposomes may contain other lipid components, as long as these do not induce instability and/or aggregation and/or chromatographic separation. This can be determined by routine experimentation.

The biocompatible hydrophilic polymer may be physically attached to the surface of the liposome, or inserted into the membrane of the liposome. The polymer may therefore be covalently bound to the liposome.

Either or both of the colloidal particle or the biologically active polypeptide or protein may be modified in order to modulate the dynamics of the association between the colloidal particle and the polypeptide or protein. Such modulation may be achieved by customising regions or binding sequences on the colloidal particle or the biologically active polypeptide or protein.

A variety of methods for producing the modified liposomes which are unilamellar or multilamellar are known and available (see Lichtenberg and Barenholz, (1988)):
1. A thin film of the phospholipid is hydrated with an aqueous medium followed by mechanical shaking and/or ultrasonic irradiation and/or extrusion through a suitable filter;
2. Dissolution of the phospholipid in a suitable organic solvent, mixing with an aqueous medium followed by removal of the solvent;
3. Use of gas above its critical point (i.e., freons and other gases such as CO2 or mixtures of CO2 and other gaseous hydrocarbons) or
4. Preparing lipid detergent mixed micelles then lowering the concentration of the detergents to a level below its critical concentration at which liposomes are formed.

In general, such methods produce liposomes with heterogeneous sizes from about 0.02 to 10 μm or greater. Since liposomes which are relatively small and well defined in size are preferred for use in the present invention, a second processing step defined as “liposome down-sizing” can be used for reducing the size and size heterogeneity of liposome suspensions.

The liposome suspension may be sized to achieve a selective size distribution of vesicles in a size range less than about 5 μm, for example <0.4 μm. In one embodiment of the invention, the colloidal particles have an average particle size diameter of from about 0.03 to 0.4 microns (μm), suitably around 0.1 microns (μm).

Liposomes in this range can readily be sterilized by filtration through a suitable filter. Smaller vesicles also show less of a tendency to aggregate on storage, thus reducing potentially serious blockage or plugging problems when the liposome is injected intravenously or subcutaneously. Finally, liposomes which have been sized down to the submicron range show more uniform distribution.

Several techniques are available for reducing the sizes and size heterogeneity of liposomes, in a manner suitable for the present invention. Ultrasonic irradiation of a liposome suspension either by standard bath or probe sonication produces a progressive size reduction down to small unilamellar vesicles (SUVs) between 0.02 and 0.08 μm in size.

Homogenization is another method which relies on shearing energy to fragment large liposomes into smaller ones. In a typical homogenization procedure the liposome suspension is recirculated through a standard emulsion homogenizer until selected liposome sizes, typically between about 0.1 and 0.5 μm are observed. In both methods, the particle size distribution can be monitored by conventional laser-beam particle size determination.

Extrusion of liposomes through a small-pore polycarbonate filter or equivalent membrane is also an effective method for reducing liposome sizes down to a relatively well-defined size distribution whose average is in the range between about 0.02 and 5 μm, depending on the pore size of the membrane.

Typically, the suspension is cycled through one or two stacked membranes several times until the desired liposome size distribution is achieved. The liposome may be extruded through successively smaller pore membranes to achieve a gradual reduction in liposome size.

Centrifugation and molecular sieve chromatography are other methods which are available for producing a liposome suspension with particle sizes below a selected threshold less than 1 μm. These two respective methods involve preferential removal of large liposomes, rather than conversion of large particles to smaller ones. Liposome yields are correspondingly reduced.

The size-processed liposome suspension may be readily sterilized by passage through a sterilizing membrane having a particle discrimination size of about 0.4 μm, such as a conventional 0.45 μm depth membrane filter. The liposomes are stable in lyophilized form and can be reconstituted shortly before use by taking up in water.

Suitable lipids for forming liposomes are described above. Suitable examples include but are not limited to phospholipids such as dimirystoylphosphatidylcholine (DMPC) and/or dimirystoyl—phosphatidylglycerol (DMPG), egg and soybean derived phospholipids as obtained after partial or complete purification, directly or followed by partial or complete hydrogenation.

The following four methods are described in WO 95/04524 and are generally suitable for the preparation of the colloidal particles (liposomes) used in accordance with the present invention.

Method A
a) mixing amphipathic substances, such as lipids suitable for forming vesicles in water-immiscible organic solvents
b) removing of the solvent in presence of a solid support, alternatively, dried amphipathic substances or mixtures thereof can be used in any form (powder, granular, etc.) directly,
c) taking up the product of step b) into a solution of the biopolymeric substances in a physiologically compatible solution
d) adding an organic solvent having solubilizing or dispersing properties, as well as
e) drying the fraction obtained in step d) under conditions retaining the function of the biopolymeric substances.

According to step a) of Method A amphipathic substances suitable for forming vesicles as mentioned above are mixed in a water-immiscible organic solvent. The water-immiscible organic solvent may be a polar-protic solvent such as fluorinated hydrocarbons, chlorinated hydrocarbons and the like.

In step b) of the method of the invention the solvent is removed in presence of a solid support. The solid support may be an inert organic or inorganic material having a bead-like structure. The material of the inorganic support material may be glass and the organic material can be Teflon™ or other similar polymers.

The step c) of Method A of the invention is for taking up the product of step b) into a solution of the substances to be encapsulated in a physiologically compatible solution.

The physiological compatible solution may be equivalent to a sodium chloride solution up to about 1.5 by weight. It is also possible to use other salts as long as they are physiologically compatible e.g. as a cryoprotectant e.g., sugars and/or amino acids. For example, lactose, sucrose or trehalose may be used as a cryoprotectant.

Optionally, between step a) and b) a step of virus inactivation, sterilizing, depyrogenating, filtering the fraction or the like of step a) can be provided. This might be advantageous in order to have a pharmaceutically acceptable solution at an early stage of the preparation.

The step d) of the Method A is adding an organic solvent having solubilizing or dispersing properties.

The organic solvent may be an organic polar-protic solvent miscible with water. Lower aliphatic alcohols having 1 to 5 carbon atoms in the alkyl chain can also be used, such as tertiary butanol (tert-butanol).

Optionally, subsequent to step d), virus inactivation sterilizing and/or portioning of the fraction yielded after step d) can be carried out.

The step e) of the present invention is drying the fraction obtained in step d) under conditions retaining the function of the substance to be loaded. One method for drying the mixture is lyophilization. The lyophilization may be carried out in presence of a cryoprotectant, for example, lactose or other saccharides or amino acids. Alternatively, evaporation or spray-drying can be used.

The dried residue can then be taken up in an aqueous medium prior to use. After taking up of the solid it forms a dispersion of the respective liposomes. The aqueous medium may contain a saline solution and the dispersion formed can optionally be passed through a suitable filter in order to down size the liposomes if necessary. Suitably, the liposomes may have a size of 0.02 to 5 μm, for example in the range of <0.4 μm.

The compositions of the invention can also be an intermediate product obtainable by isolation of either fraction of step c) or d) of the method A. Accordingly, the formulation of the invention also comprises an aqueous dispersion obtainable after taking up the product of step e) of method A in water in form of a dispersion (liposomes in aqueous medium).

Alternatively, the pharmaceutical compositions of the invention are also obtainable by the following methods which are referred to as Methods B, C, D and E.

### Method B

This method comprises also the steps a), b) and c) of the Method A. However, step d) and e) of Method A are omitted.

### Method C

In Method C step d) of method A is replaced by a freeze and thaw cycle which has to be repeated at least two times. This step is well-known in prior art to produce liposomes.

### Method D

Method D excludes the use of any osmotic component. In method D the steps of preparation of vesicles, admixing and substantially salt free solution of the substances to be loaded and co-drying of the fractions thus obtained is involved.

### Method E

Method E is simpler than methods A-D described above. It requires dissolving the compounds used for liposome preparation (lipids antioxidants, etc.) in a polar-protic water miscible solvent such as tert.-butanol. This solution is then mixed with an aqueous solution or dispersion containing the blood factor. The mixing is performed at the optimum volume ratio required to maintain activity.

The mixture is then lyophilized in the presence or absence of cryoprotectant. Rehydration is required before the use of the liposomal formulation. These liposomes are multilamellar, their downsizing can be achieved by one of the methods described in WO 95/04524.

As used herein, the term “treatment” includes any regime that can benefit a human or a non-human mammal. The treatment of “non-human mammals” extends to the treatment of domestic mammals, including horses and companion animals (e.g. cats and dogs) and farm/agricultural animals including members of the ovine, caprine, porcine, bovine and equine families. The treatment may be in respect of any existing condition or disorder, or may be prophylactic (preventive treatment). The treatment may be of an inherited or an acquired disease. The treatment may be of an acute or chronic condition.

Levels of activity in the blood coagulation cascade may be measured by any suitable assay, for example the Whole Blood Clotting Time (WBCT) test or the Activated Partial Thromboplastin Time (APTT).

The Whole Blood Clotting Time (WBCT) test measures the time taken for whole blood to form a clot in an external environment, usually a glass tube or dish.

The Activated Partial Thromboplastin Time (APTT) test measures a parameter of part of the blood clotting pathway. It is abnormally elevated in haemophilia and by intravenous heparin therapy. The APTT requires a few millilitres of blood from a vein. The APTT time is a measure of one part of the clotting system known as the “intrinsic pathway”. The APTT value is the time in seconds for a specific clotting process to occur in the laboratory test. This result is always compared to a “control” sample of normal blood. If the test sample takes longer than the control sample, it indicates decreased clotting function in the intrinsic pathway. General medical therapy usually aims for a range of APTT of the order of 45 to 70 seconds, but the value may also be expressed as a ratio of test to normal, for example 1.5 times normal. A high APTT in the absence of heparin treatment can be due to haemophilia, which may require further testing.

The invention also provides a kit of parts comprising a composition of the invention, and an administration vehicle including injectable solutions for administration, said kit suitably comprising instructions for use thereof.

The invention therefore may also suitable provide a dosage form of a composition of the invention. Such dosage forms may be provided as suitable containers or vials containing the appropriate dose for a patient.

A dosage up to 2.000 mg/liposomes lipid per kg body weight can be administered to patients.

Accordingly, in another aspect of the present invention, the volume of the formulation for delivery into a patient may be no more than 2 ml. Suitably, the delivery volume may be 5 μl, 10 μl, 25 μl, 50 μl, 100 μl, 250 μl, 500 μl, 750 μl, or 1 ml. In alternative embodiments the volume of the formulation for delivery may be no more than 1.5 ml, 2 ml, 2.5 ml, 3.0 ml or 3.5 ml.

The formulations of the invention may be for administration at least once per day, at least twice per day, about once per week, about twice per week, about once per two weeks, or about once per month.

The composition comprising colloidal particles for use according to the invention may be formulated for administration by any convenient route, such as subcutaneous, intravenous or topical administration. The colloidal particles may therefore be formulated as a pharmaceutical composition, wherein the composition does not contain any pharmaceutically active agent.

A formulation suitable for topical, subcutaneous or intravenous administration may suitably be prepared as an aqueous or substantially aqueous formulation. The formulation may comprise such additional salts, preservatives and stabilisers and/or excipients or adjuvants as required. The dosage forms of the invention may be provided as anhydrous powders ready for extemporaneous formulation in a suitable aqueous medium.

Suitably such dosage forms can be formulated as buffered aqueous formulations. Suitable buffer solutions may include, but are not limited to amino acids (for example histidine), salts of inorganic acids and alkali metals or alkaline earth metals, (for example sodium salts, magnesium salts, potassium salts, lithium salts or calcium salts—exemplified as sodium chloride, sodium phosphate or sodium citrate). Other components such as detergents or emulsifiers (for example, Tween 80® or any other form of Tween®) may be present and stabilisers (for example benzamidine or a benzamidine derivative). Excipients such as sugars, (for example sucrose) may also be present. Suitable values for pH are physiological pH, e.g. pH 6.8 to 7.4 or pH 7.0. The pH may be adjusted accordingly with a suitable acid or alkali, for example hydrochloric acid. Liquid dosage forms may be prepared ready for use in such administration vehicles, for example as 3.5 ml or 7.0 ml vials.

In one particular embodiment, there is provided a composition for intravenous or subcutaneous administration for use in accordance with the invention as follows:
50 mM sodium citrate
pH 7.0
100 mM phospholipids—97:3 molar ratio of palmitoyl-oleoyl phosphatidyl choline (POPC) and 1,2-distearoyl-sn-glycero-3phosphoethanol-amine-N-[poly-(ethyleneglycol)-2000] (DSPE-PEG 2000).

Formulations for topical administration may be formulated using a topical gel comprising one or more components selected from the group consisting of: surfactants, preservatives, thickeners, buffers, and water. In one embodiment, the surfactant may be a nonionic surfactant selected from the group consisting of: polyoxyethylene sorbitans, polyhydroxyethylene stearates or polyhydroxyethylene laurylethers, optionally the surfactant is polysorbate 80 (Tween 80). The ratio of phospholipid to surfactant may be from 30:1, to 15:1, 10:1, suitably 15:1, 8:1, or 2:1. The surfactant concentration may be from 0.25% to 5% by weight, for example 1% to 3%, 1 to 2%, some exemplary values may be 0.47%, 0.85%, or 3.5%. Examples of such formulations for topical administration are described in WO 2010/140061 and WO 2011/022707.

The invention will now be further described by way of reference to the following examples which are present for the purposes of illustration only and are not be taken as limitations to the invention:

### EXAMPLE 1: SYNTHESIS OF LIPOSOMES

Mixed lipids were prepared from palmitoyl-oleoyl phosphatidyl choline (POPC) and 1,2-distearoyl-sn-glycero-3-phosphoethanol-amine-N-[poly-(ethyleneglycol)-2000] derivatized with PEG-2000 (PEG with molecular weight 2000 Daltons) (DSPE-PEG 2000), as follows:
Molecular weight of POPC: 760.08 g/mol
Molecular weight of DSPE-2kPEG: 2789.5 g/mol

The final preparation had a concentration of 100 mM phospholipids. A 15% w/v mixture of lipids was made with a 97:3 molar ratio of POPC:DSPE-2kPEG. The following were weighed and mixed:
2.04 g POPC
0.232 g DSPE-2kPEG
14.9 mL tert-butanol (melted in a 35° C. water bath), all placed in a 100 mL Schott bottle.

The mixture was maintained at 35° C. in a water bath and stirred intermittently until all solids had dissolved/dispersed. The final material was a clear colourless mixture. The mixture was frozen at −80° C. overnight.

The operation was maintained in a fume hood to allow containment during the post-use clean-up of dried/condensed solvent. The Christ Alpha 1-2 LD freeze-drier and vacuum pump were warmed up for 20 minutes, and the frozen lipid/solvent mixture was removed from −80° C. storage and dried overnight.

The dried lipids were recovered from the drier the following morning. They appeared as a dry crystalline cake. A 100 mM lipid solution was required for further processing. The quantities of lipid present calculate through as around 82 μmoles of DSPE-2kPEG and 2.69 mmoles of POPC; so around 2.77 mmoles of lipids. Thus 27.7 mL of diluent was required. 27.7 mL of 50 mM sodium citrate buffer was added to the dried lipids, and the resulting mixture was stirred and heated to around 35° C. After around 120 minutes, a white emulsion with no obvious large solids resulted. This was subjected to extrusion as below.

A Sartorius 47 mm stainless steel pressure filtration housing was assembled and wrapped with a water jacket (wrapped tubing fed via a thermocirculator) maintained at 35° C. The housing was fitted with a polycarbonate track-etched membrane (details below), covered by a glass-fibre prefilter (Whatman GF/D). The emulsion was poured into the housing and extruded under 4 bar nitrogen gas, with the filtrate collected into 50 mL tubes. The duration of each extrusion was timed and noted.

The filtration sequence was: 0.8 μm, 0.4 μm, 0.2 μm, 0.2 μm, 0.1 μm and 0.1 μm (i.e. single passes through the larger filters and two passes through the smaller 0.2 and 0.1 μm filters), with the filtrate warmed back to 35° C. between passes. The liposomes were extruded, with tabulated data is below:

TABLE 1

| Pore | | | Recovery |
|---|---|---|---|
| size (μm) | Duration | | (g) |
| 0.8 | <4 | sec | 28.19 |
| 0.4 | <4 | sec | 26.91 |
| 0.2 | 50 | sec | 23.76 |
| 0.2 | 22 | sec | 21.77 |
| 0.1 | 12 | minutes | 20.18 |
| 0.1 | 4 | minutes | 19.47 |

The resulting extruded lipids were stored at +5° C. 15 mL of ‘Extruded Liposomes’ were removed from the chilled stock and dispensed into a sterile 50 mL tube within a MicroBiological Safety Cabinet. The size of the extruded liposomes was analysed using an ALV5000 photon correlation spectrometer. The average radius was determined to be 75.40±0.86 nm and the average peak width 22.21±3.86 nm, giving an average diameter of 150.80 nm and polydispersity index of 0.087.

### EXAMPLE 2: FORMULATION OF PEGYLATED LIPOSOMES FOR TOPICAL ADMINISTRATION

PEGylated Liposomes in citrate buffer were produced in accordance with Example 1 above according to the method of Baru et al. (2005). The Liposome formulation had the following composition; 50 mM sodium citrate pH 7.0 containing 100 mM phospholipids; comprising a 97:3 molar ratio mixture of palmitoyl-oleoylphosphatidylcholine (POPC) and 1,2-distearoyl-sn-glycero-3-phosphoethanol-amine-N-[poly-(ethyleneglycol)-2000] (DSPE-PEG 2000).

Exemplary topical formulations may be prepared according to the following:

TABLE 2

| | Percentage; |
|---|---|
| Ingredient | g per 100 g |
| Soy Phosphatidylcholine | 6.680 |
| Polysorbate 80 (Tween ® 80) | 0.850 |
| Benzylalcohol | 0.525 |
| Methyl-4-hydroxybenzoate | 0.250 |
| Ethyl-4-hydroxybenzoate | 0.250 |
| Butylhydroxytoluene | 0.020 |
| Linalool | 0.100 |
| mPEG-DSPE-2,000 Mw | 0.760 |
| Disodium EDTA | 0.100 |
| Disodium hydrogen | 0.061 |
| orthophosphate 12 H₂O | |
| Sodium dihydrogen | 0.755 |
| orthophosphate 2 H2O | |
| Glycerol | 3.000 |
| Ethanol * | 3.081 |
| Sodium hydroxide | 0.630 |
| Carbopol 974P NF | 1.250 |
| Water | 81.688 |
| Total | 100.000 |
| pH | 7.5 |

### EXAMPLE 3: PEGYLATED LIPOSOMES FOR TOPICAL ADMINISTRATION AND TREATMENT OF MILD TO MODERATE HAEMOPHILIA A

The test subject is dosed in order to potentiate the effects of endogenous Factor VIII in mild to moderate haemophilia A.

Following dosing of the subject with the PEGylated liposomes, the test subject is observed for clinical signs. Unexpected toxicities are screened for by performing CBC and serum chemistry tests at 48 hr and 5 days post-dose. Fibrinogen, FDPs and the thrombin time (TT) are evaluated to test for increased thrombosis risk.

Blood samples (5 ml) are taken from the subject dosed SQ at the following times points after administration:
Pre-drug administration and at 0.5, 1, 2, 4, 8, 12, 24, 36, 48, 60, 72, 84, 96, 108 and 120 hours post-dose.

Whole blood (non-citrated; 1 ml) is used for the whole blood clotting assay and the activated clotting time assay. The remaining 4 ml blood samples are transferred into tubes containing 0.109M tri-sodium citrate anticoagulant (9:1 v/v) on ice.

The activated Partial Thromboplastin Time (aPTT), Activated Clot Time (ACT) and Thromboelastogram (TEG) assays are conducted on the citrated whole blood.

Plasma is prepared by centrifugation of the remaining citrated blood and the resulting plasma samples are stored in aliquots of approximately 100 μl at −80° C.

### Assays

### (i) Non-Citrated Whole Blood: Whole Blood Clotting Assay

Blood samples are divided between 2 vacutubes, (2×0.5 ml) and observed carefully with periodic and judicious levelling of the tube until a clot was determined by interruption of flow in the fully horizontal position. The quality of the clot is observed by holding the tube in the fully inverted position. The whole blood clotting time was recorded as the mean of the total time from sample extraction until visual observation of blood clot for both samples and the quality of the clot in the inverted position is noted.

### (ii) Citrated Whole Blood: Thromboelastogram (TEG) Assav

TEG is performed with re-calcified citrated whole blood using a Hemostasis Analyzer Model 5000 (Haemoscope Corporation) thromboelastograph according to the manufacturers' recommendations. Briefly, 1 ml of citrated whole blood is placed in a commercially available (Teg® Hemostasis System Kaolin, Haemonetics) vial containing kaolin. Mixing is ensured by gentle inversion of the kaolin-containing vials 5 times. Pins and cups are placed in the TEG analyzer in accordance with the standard procedure recommended by the manufacturer. Each standard TEG cup is placed in the 37° C. pre-warmed instrument holder and is filled with 20 μl of calcium chloride (0.2 M). Then, 340 μl of kaolin-activated citrated whole blood is added for a total volume of 360 μl.

(iii) Activated Clotting Time (ACT) and Activated Partial Thromboplastin Time (aPTT)

The ACT and aPTT tests are carried out using a Haemachron Jr coagulation analyzer (International Technidyne Corps.) according to the manufacturer's instructions.

### (iv) Plasma: FVIII Activity Assay (Chromogenic)

FVIII plasma activity is determined using the Coatest Assay (Dia Pharma, West Chester, Ohio). Plasma samples are diluted 1:20 to 1:80 with assay diluent and assayed according to the manufacturer's instructions. Standard curves are established using normal hemostasis reference plasma (American Diagnostica Inc, Stamford, Conn.) and the purified FVIII protein.

### (v) Plasma: FVIII ELISA

The concentration of FVIII antigen in plasma samples is determined by ELISA using the Visulize FVIII antigen kit from Affinity Biologicals (Ancaster, Ontario, Canada) according to the manufacturer's instructions.

### (vi) Plasma: Immunogenicity

Bethesda assays are conducted on 1:4, 1:10 and 1:20 dilutions of test plasma into FVIII deficient human plasma. Equal volumes of the diluted test plasma and normal human reference plasma are incubated at 37° C. for 2 hours and the Bethesda titre determined using the aPTT assay and a normal human plasma standard curve as described above.

### EXAMPLE 4: FORMULATION OF PEGYLATED LIPOSOMES FOR INTRAVENOUS (IV) OR SUBCUTANEOUS (SQ) ADMINISTRATION

PEGylated Liposomes in citrate buffer were produced in accordance with Example 1 above according to the method of Baru et al. (2005) comprising a mixture of palmitoyl-oleoylphosphatidylcholine (POPC) and 1,2-distearoyl-sn-glycero-3-phosphoethanol-amine-N-[poly-(ethyleneglycol)-2000] (DSPE-MPEG 2000).

Exemplary SQ or IV formulations may be prepared according to the following:

TABLE 3

| | SQ/IV | SQ/IV | SQ/IV |
|---|---|---|---|
| | Formulation | Formulation | Formulation |
| | 9% Total | 7% Total | 4% Total |
| | Lipids | Lipids | Lipids |
| Substance | (mg/mL) | (mg/mL) | (mg/mL) |
| 1-Palmitoyl-2- | 81 | 63 | 36 |
| oleoyl-sn- | | | |
| glycero-3- | | | |
| phosphocholine | | | |
| (POPC) | | | |
| N-(Carbonyl- | 9 | 7 | 4 |
| methoxypoly- | | | |
| ethylene- | | | |
| glycol-2000)- | | | |
| 1,2-distearoyl- | | | |
| sn-glycero-3- | | | |
| phospho- | | | |
| ethanolamine, | | | |
| sodium salt | | | |
| (MPEG-2000- | | | |
| DSPE) | | | |
| Sodium citrate | 9 | 9 | 9 |
| Hydrochloric acid | q.s. (1) | q.s. (1) | q.s. (1) |
| Water | q.s. (2) | q.s. (2) | q.s. (2) |
| Total | 1000 | 1000 | 1000 |

q.s.(1)-Quantity sufficient to adjust pH to 6.
q.s.(2)-Quantity sufficient to adjust volume to 1 mL

### EXAMPLE 5: GEL AND SPRAY FORMULATIONS OF PEGYLATED LIOSOMES FOR TOICAL ADMINISTRATION WITH ALTERED LEVELS OF SURFACTANT

PEGylated Liposomes in citrate buffer were produced in accordance with Example 1 above according to the method of Baru et al. (2005) comprising a mixture of soy phosphatidylcholine (SPC) and 1,2-distearoyl-sn-glycero-3-phosphoethanol-amine-N-poly-(ethyleneglycol)-2000] (DSP)-MP-G2000). The formulations were prepared to test different physical forms as a gel or spray for topical administration.

TABLE 4

| | Topical | Topical | Topical | Topical |
|---|---|---|---|---|
| | formulation | formulation | formulation | formulation |
| | (0.85% | (3.5% | (3.5% | (0.47% |
| | surfactant) | surfactant)- | surfactant)- | surfactant) |
| Substance | (mg/mL) | spray (mg/mL) | gel (mg/mL) | (mg/mL) |
| Soy | 68.700 | 64.516 | 64.516 | 71.460 |
| Phosphatidylcholine | | | | |
| Polysorbate 80 | 8.500 | 35.484 | 35.484 | 4.720 |
| N-(Carbonyl- | 7.6 to 9.0 | 7.6 to 9.0 | 7.6 to 9.0 | 7.6 to 9.0 |
| methoxypoly- | | | | |
| ethyleneglycol- | | | | |
| 2000)-1,2- | | | | |
| distearoyl-sn- | | | | |
| glycero-3- | | | | |
| phospho- | | | | |
| ethanolamine, | | | | |
| sodium salt | | | | |
| (MPEG-2000- | | | | |
| DSPE) | | | | |
| Benzyl alcohol | 5.250 | 5.250 | 5.250 | 5.250 |
| Methylparaben | 2.500 | 2.500 | 2.500 | 7.500 |
| Ethylparaben | 2.500 | 1.700 | 1.700 | 0.000 |
| Propylparaben | 0.000 | 0.000 | 0.000 | 2.500 |
| Butylhydroxyanisol | 0.000 | 0.200 | 0.200 | 0.200 |
| Butylhydroxytoluol | 0.200 | 0.000 | 0.000 | 0.000 |
| Linalool | 1.000 | 1.000 | 1.000 | 1.000 |
| Sodium | 0.500 | 0.000 | 0.000 | 0.000 |
| metabisulphite | | | | |
| Disodium edetate | 1.000 | 3.000 | 3.000 | 3.000 |
| diSodium | 7.550 | 7.720 | 7.720 | 7.720 |
| hydrogen | | | | |
| orthophosphate | | | | |
| dodecahydrate | | | | |
| Sodium | 0.610 | 4.440 | 4.440 | 4.440 |
| dihydrogen | | | | |
| orthophosphate | | | | |
| dihydrate | | | | |
| Glycerol | 30.000 | 30.000 | 30.000 | 50.000 |
| Ethanol | 36.510 | 30.000 | 30.000 | 30.000 |
| Sodium | 6.300 | 0.000 | 0.000 | 0.000 |
| hydroxide | | | | |
| Trometamol | 0.000 | 0.000 | 12.677 | 0.000 |
| (TRIS) | | | | |
| Carbopol 974P | 12.500 | 0.000 | 10.000 | 0.000 |
| NF | | | | |
| Water | q.s. to 1 mL | q.s. to 1 mL | q.s. to 1 mL | q.s. to 1 mL |
| Total | 1000.000 | 1000.000 | 1000.000 | 1000.000 |

q.s.-Quantity sufficient to adjust volume to 1 mL.

1-25. (canceled)

26. A method of treating a patient suffering from haemophilia A comprising administering to said patient a composition consisting of a colloidal particle; said colloidal particle consisting of about 0.5 to 20 mole percent of a first amphipathic lipid derivatized with a biocompatible hydrophilic polymer and a second amphipathic lipid, wherein the composition is formulated as a buffered aqueous formulation.

27. A method of treating a patient suffering from haemophilia A comprising administering to said patient a composition consisting of a colloidal particle; said colloidal particle consisting of about 0.5 to 20 mole percent of a first amphipathic lipid derivatized with a biocompatible hydrophilic polymer, a second amphipathic lipid and a non-ionic surfactant, wherein the composition is formulated as a buffered aqueous formulation.

28. The method of claim 26 or claim 27, wherein the colloidal particles are substantially neutral and the polymer carries substantially no net charge.

29. The method of claim 26 or claim 27, wherein the colloidal particle has a mean particle diameter of between about 0.03 to about 0.4 microns (μm).

30. The method of claim 29, wherein the colloidal particle has a mean particle diameter of approximately 0.1 microns (μm).

31. The method of claim 26 or claim 27, wherein said first amphipathic lipid is a phospholipid from natural or synthetic sources.

32. The method of claim 31, wherein said first amphipathic lipid is a phosphatidylethanolamine (PE).

33. The method of claim 26 or claim 27, wherein said first amphipathic lipid is a carbamate-linked uncharged lipopolymer.

34. The method of claim 33, wherein said first amphipathic lipid is aminopropanediol distearoyl (DS).

35. The method of claim 26 or claim 27, wherein the second amphipathic lipid is obtained from either natural or synthetic sources.

36. The method of claim 35, wherein the second amphipathic lipid is a phosphatidylcholine (PC).

37. The method of claim 36, wherein the colloidal particle consists of palmitoyl-oleoyl phosphatidyl choline (POPC) and 1,2-distearoyl-sn-glycero-3-phosphoethanol-amine (DSPE) in a ratio (POPC:DSPE) of from 85 to 99:15 to 1.

38. The method of claim 37, wherein the ratio of POPC:DPSE is from 90 to 99:10 to 1.

39. The method of claim 38, wherein the ratio of POPC:DPSE is 97:3.

40. The method of claim 26 or claim 27, wherein said biocompatible hydrophilic polymer is selected from the group consisting of polyalkylethers, polylactic acids and polyglycolic acids.

41. The method of claim 40, wherein said biocompatible hydrophilic polymer is polyethylene glycol.

42. The method of claim 41, wherein the polyethylene glycol has a molecular weight of between about 500 to about 5000 Daltons.

43. The method of claim 42, wherein the polyethylene glycol has a molecular weight of approximately 2000 Daltons.

44. The method of claim 41, wherein the derivatized amphipathic lipid is 1,2-distearoyl-sn-glycero-3-phosphoethanol-amine-N-[poly-(ethyleneglycol)].

45. The composition of claim 41, wherein the derivatized amphipathic lipid is 1,2-distearoyl-sn-glycero-3-phosphoethanol-amine-N-[poly-(ethyleneglycol)-2000] (DSPE-PEG 2000).

46. The method of claim 26, wherein the composition is formulated for intravenous or subcutaneous administration.

47. The method of claim 27, wherein the non-ionic surfactant is selected from the group consisting of polyoxyethylene sorbitans, polyhydroxyethylene stearates or polyhydroxyethylene laurylethers.

48. The method of claim 27 wherein the composition is formulated for topical, intravenous or subcutaneous administration.
